(19) United States
(12) Patent Application Publication
(10) Pub. No.: US 2009/0270859 A1
(43) Pub. Date: Oct. 29, 2009

(54) FLUID COMPOSITIONS AND METHODS FOR THE USE THEREOF

(76) Inventor: Ari Hirvi, Riihimaki (FI)

Correspondence Address: SMITH-HILL AND BEDELL, P.C., 16100 NW CORNELL ROAD, SUITE 220, BEAVERTON, OR, 97006, US

(21) Appl. No.: 12/407,194

(22) Filed: Mar. 19, 2009

Related U.S. Application Data
(60) Provisional application No. 61/038,117, filed on Mar. 20, 2008.

(51) Int. Cl. A61B 18/18; A61J 1/10; A61K 47/02; A61K 31/407

(52) U.S. Cl. 606/48; 604/410; 514/769; 514/410

(57) ABSTRACT

A method of performing transurethral surgery on the human or animal body. A tissue is cut using a first active cutting electrode and a second neutral electrode, both electrodes being immersed in an electrically conductive aqueous fluid and connected to a power source for applying a voltage and a current between the electrodes. According to the invention, the electrical conductivity of the fluid is at least 25 mS/cm at room temperature. With the invention, complications of surgery can be significantly reduced.

### BACKGROUND OF THE INVENTION

1. Field of the Invention

The present invention relates to electrically conductive fluid compositions and to methods for the use thereof. In particular, the present invention concerns fluid compositions that can be used as irrigation fluids for example in transurethral and transcutaneus surgery and cancer therapy and diagnostic procedures, as well as surgical and diagnostic procedures performed through natural or for the purpose made body orifices where either cutting or coagulating electric current or plasmakinetic effect produced by electricity is being used.

Surgery and diagnostic procedures performed to humans as well as veterinary and dental surgery and diagnostic procedures are covered alike.

2. Description of Related Art

Transurethral and transcutaneus surgery and diagnostic procedures have traditionally been performed with resectoscope loops and, more recently, using a laser-cutting technique known as HF-resection, wherein adenoma tissue proceeding from the sphincter has been peeled out in the direction of the neck of the bladder with a laser.

As a conductive liquid, only sterile water and saline which contains 9 mg of sodium per 1 ml water has been used. This is due to a unfounded idea that only close to physiological solutions can and should be used. An idea that we will prove to be wrong: the urothelium lining the upper urinary track, the bladder and the urethra is able to withstand without harm great variations in the composition and osmolality of both urine and irrigation fluids. The use of non-physiological solutions will probably be advantageous in most endourological therapies.

Further improvements comprise the use dissection electrodes. Thus, in the so-called mono-polar technique, tissue cutting is being performed by a very hot cutting loop or spike or other suitably shaped instrument (U.S. Pat. No. 6,447,509, Wolff) placed at the end of a resectoscope where the heat is generated by high frequency (HF) directed current (DC) that is being let through the loop or other instrument at the end of the resectoscope at the will of the operator. The loop or other instrument is then moved axially to and fro by a moving a handle mechanism operated by the surgeon's hand. The reciprocating movement is coordinated with swiveling of the shaft of the resectoscope and, by proceeding in the indicated way, pieces of desired thickness and length are cut from the patient's tissue on which the diagnostic or curative operation is being done.

The loop or the other instrument forms the active electrode and the electric current is been led through the patient's tissues to a grounding electrode placed on the patient's skin which forms the neutral electrode. A voltage gradient is created at the said loop or other instrument, this gradient causes the loop, or other instrument, and the tissue that comes into contact with it to heat, and cutting of the tissue becomes possible. By alternating the qualities of the HF current a coagulating effect on the blood vessels is also achieved.

The operations, inside either the bladder or in a cavity in the prostate or inside other natural or artificially made body cavity, have been made until very recently with tissue irrigation done with a non-conducting fluid, since a conducting fluid would disperse the current and prevent the generation of the voltage gradient needed to cut the tissue.

The traditional method has several drawbacks as far as patient safety is concerned. Thus, the flow of current through the patient's tissues that are not to be operated has been known to cause, depending on the type of operation, excessive bleeding, impotence, urinary incontinence and in some cases the perforation of the bladder and vessels and seeding tumor cells outside the bladder rendering it potentially uncurable by excisions and other local therapy. The perforation often happens when the so called “obturator twitch” has occurred i.e. the near bladder wall lying obturator nerve is irritated by the current and an rapid and powerful contraction of those thigh muscles that the said nerve innervates happens and the patient's pelvic area and/or leg jerks suddenly and the cutting instrument may perforate the bladder wall.

Bladder perforation is the second most common reported complication after bleeding. It occurs in 1.3%-4% of mono-polar transurethral bladder resections, most being of extraperitoneal type and suitable for conservative treatment. The more serious intraperitoneal perforation occurs in 0.25%-0.5% of the cases. In a recently published study 75% of the patients who necessitated open surgery after a bladder perforation during a TUR all presented with extravesical recurrence after a mean follow-up of 7.5 months. Of the patients with extravesical relapse 75% died of the disease (Skolarikos A, Ghristifos M, Ferakis N, Papatsoris A, Dellis A, deliveliotis C: J Urol 2005 June; 173(6):1908-1911).

It is possible to prevent the obturator twich using special anesthetic techniques like a selective preoperative obturator nerve block when neuraxial blockade with epidural or spinal anaesthesia is chosen, but this is technically demanding and not always 100% successful: a life-threatening massive hemorrhage following obturator artery injury during transurethral bladder surgery due to violent leg jerking has been reported. The injury was caused by the resectoscope associated with the bladder perforation.

A study of the literature by the authors of said report suggested that both the nerve stimulation technique and anatomical approach used were appropriate. It was therefore unlikely that the block resulted in failure because of an inappropriate site for deposition of the anaesthetic. Consensus does not appear to have been obtained as to the concentration and volume of the anesthetic necessary for prevention of the obturator nerve stimulation during the transurethral procedures.

General anesthesia in combination with an intravenous muscle relaxation with d-tubocurarine and succinylcholine and related substances is another long known and widely used technique.

However, it is preferable to use neuraxial blockade with epidural or spinal anaesthesia as anesthesia during operations because the risk of serious anesthesia complications and even death is much higher when general anesthesia is used, especially in geriatric population. Postoperative complications are also much lower with the use of neuraxial blockade. In an overview of randomised trials the overall mortality was reduced by about a third in patients allocated to neuraxial blockade. Neuraxial blockade reduced the odds of deep vein thrombosis by 44%, pulmonary embolism by 55%, transfusion requirements by 50%, pneumonia by 39%, and respiratory depression by 59% (all P<0.001). There were also reductions in myocardial infarction and renal failure [Rodgers A, Walker N, Schug S, McKee A, Kehlet H, van Zundert A, Sage D, Futter M, Saville G, Clark T, MacMahon S: BMJ. 2000 Dec. 16; 321(7275): 1493].

About 25% of anesthesia-related deaths are directly associated with technical failures in general anesthesia [Hove L D, Steinmetz J, Christoffersen J K, Møller A, Nielsen J, Schmidt H: Anesthesiology 2007 April; 106(4):675-80].

Spinal anesthesia has been shown to be as good as general anesthesia even in heavy urological surgery like radical prostatectomy. Spinal anesthesia is overall a much safer choice to the patient and it should always be used in surgery when it is applicable.

One of the feared and sometimes lethal complications is the TUR-syndrome, where the non-conducting fluid is absorbed in the patient during prostate operations. The hyponatremia and excess of ammonia in the blood caused by this will cause symptoms such as bradycardia, confusion, tachypnea, convulsions, anesthesia problems, coma and sometimes death if not observed early and decisive and rapid action is taken, which often requires referring the patient to the intensive care unit.

The risk of the TUR-syndrome has been somewhat reduced by the use of L-arginine in the irrigation fluid (GB Patent Specification No. 2 234 897 A) and by adding mannitol to the fluid (U.S. Pat. No. 2,721,825) which increases the rate at which the absorbed fluid is eliminated by patient's kidneys.

The most common system used to monitor the amount of irrigation fluid absorbed by the patient is to add some ethyl alcohol in the irrigation fluid; the amount of alcohol in the patient's blood circulation then being measured from exhaled air using a breath alcohol meter that selectively analyzes the alcohol concentration in units of ppm in human breath.

A major improvement in both the cutting effect and patient safety was achieved by the invention of bi-polar/Plasma Kinetic electrosurgical generator and system by Gyrus Medical Limited (U.S. Pat. No. 6,306,134 B1) and development and use of instruments using the bi-polar current technique by companies like ACMI, Gyrus, Olympus, Storz and Wolf as maybe the most well known.

In the bipolar technique both the active and the neutral electrode are placed so that the irrigation fluid acts as a conductor so that the amount of current passed through the patient's tissues is reduced greatly. This reflects directly on the rate of the before mentioned complications caused by the current i.e. the new technique is considered better because there is much less nerve irritation and damage in the adjoining tissues.

As a conductive liquid, saline which contains 9 mg of sodium per 1 ml water has been used. This is advantageous not only because it conducts current well, but also because absorption of it does not cause TUR-syndrome.

While normotonic irrigation fluid seems to solve most of the problems associated with TUR and similar operations and diagnostic procedures, some problems persist: it has been reported that some 10% of the current still flows through the patient's tissues (Brunken C, Qiu H, Tauber R: Urologe A 2004 September; 43(9): 1101-5) and some obturator nerve irritation takes place. There are only few reports on the long time complications of mono-polar versus bi-polar resection and there seems to be no significant difference in favour of bi-polar technique in this respect, while the short time benefits are indisputable (Starkman J S, Santucci R A: BJU Int. 2005 January; 95(1): 69-71). In two studies using two different bi-polar devices (Gyrus, Olympus) a higher rate of urethral strictures than with the monopolar technique was detected [Rassweiler J, Schulze M, Stock C, Teber D, De La rosette J: Minim Invasive Ther Allied Technol. 2007; 16(1): 11-21].

### SUMMARY OF THE INVENTION

It is an aim of the present invention to eliminate at least a part of the problems related to the known technology and to provide a novel way of performing transurethral surgery on the human or animal body.

While resection of especially the bladder is technically easier with bipolar technique and saline than using mono-polar technique, a surprisingly high occurrence of the obturator twich still persists. Since spinal anesthesia is overall a much safer choice to the patient and it should always be used, a method of improving the safety of bladder resection with bipolar technique with neuraxial blockade was developed.

A key to improving the safety is therefore to reduce the amount current flowing through the patient's tissues. Principally, the lower the current, the less current will flow through the tissue. However, a lowering of the overall current is not possible with today's equipment: the plasma kinetic (PK) effect requires rather a high effect, albeit for a short time, to fire, effects of 180-260-320 watts are being used. No plasma kinetic effect can generally be seen on the instrument when using effects below 50 watts, and cutting of tissue is therefore typically not possible under 80 watts.

The present invention is, instead, based the finding that the flow of the irrigation fluid has an important effect on the use of the equipment in transurethral surgery. For example, a rapid flow past the cutting loop or spike or instrument often prevents the PK from firing at lower power settings. So to cut the tissue a rather high effect is needed. But by adjusting the composition of the irrigation fluid, it is also possible to reduce the amount of current through the body during surgery.

Therefore, the present invention is based on the concept of considerably increasing the conductivity of the irrigation fluid. In particular, the present invention provides for the use, for example in transurethral surgery on the human or animal body, of fluids having high electrical conductivity, preferably of at least 25 mS/cm, suitably at least 28 mS/cm, or more, at room temperature.

Surprisingly, it has been found that by adjusting the concentration of monovalent cations in the fluid, it is possible greatly to increase conductivity to reduce the current through the patient's body during transurethral surgery, without still causing any adverse effects on the patient wellbeing due to the use of hypertonic liquids.

More specifically, the method according to the present invention is mainly characterized by what is stated in the characterizing part of claim 1.

The electrically conductive fluid is characterized by what is stated in the characterizing part of claim 16.

Considerable advantages are obtained by means of the invention. Since most adverse effects and post operative problems associated with electrosurgery are derived from the inappropriate flow of electricity through tissues which should spared from such exposure, such as the muscle and the nerves near the outer capsule of the prostate and the bladder, just to name some examples, the cutting down of said electricity will lessen the complications of surgery greatly.

The present invention is applicable to different kinds of methods of surgery, including transurethral surgery, such as transurethral resection of the bladder, transurethral resection and transurethral enucleating of the prostate.

However, the invention is not limited to transurethral or transcutaneous surgery or diagnosis. It is basically possible to carry out the invention using any normal body cavities or cavities purpose-made in the human or animal body. Thus, it is possible to apply the invention to surgery aiming at removing cartilage inside joints wherein conventionally bi-polar devices similar to the ones discussed above can be used.

Further, the irrigation liquids can be used in cancer therapy: subjecting the cancer cells to irrigations of varying osmolality during the treatment may well have an additive effect to other treatment modalities like mithomycin.

The present fluid can be placed in a simple storage container or in a combination bag comprising two or more compartments, wherein at least one compartment is filled with the present fluid or wherein a fluid can be formed by combining the content of two or more compartments. Examples of suitable multi chamber infusion bags can be found in U.S. Pat. No. 3,542,561, the contents of which are herewith incorporated by reference. The advantage of such bags is that the additive can be mixed into the rest of the bag immediately before the use.

Next the invention will be examined more closely with the aid of a detailed description.

### DETAILED DESCRIPTION OF PREFERRED EMBODIMENTS

As mentioned above, the present invention concern improved methods of transurethral surgery. In such methods, a tissue is typically cut using a device comprising a first active cutting electrode and a second neutral electrode, both electrodes being immersed in an electrically conductive aqueous fluid and connected to a power source for applying a voltage and a current between the electrodes.

Thus, bipolar current is used so as to minimize the magnitude of the current passing though the tissues of the patient. In the method, both electrodes of the instruments are immersed in the liquid inside the patient in a natural or artificially created cavity or an extension thereof.

The basic technology for performing bipolar surgery is disclosed in U.S. Pat. No. 6,306,134, the contents of which are herewith incorporated by reference. In order to reduce electric current through human or animal tissue, the aqueous fluid should have a conductivity of at least 25 mS/cm at room temperature. As regards tissue as a conductive path of electricity it should be pointed out that the electrical properties vary depending on the composition of the actual tissue. For example, fat is an extremely bad conductor, whereas blood is, due to its high electrolyte content, a very good conductor. The susceptibility of a patient to be injured by this stray electricity is something which may have to do with simply how much muscle or perivesical or periprostatic fat the patient has. Different tissues conduct electricity in a different manner.

Obesity may thus protect patients from the adverse effects of electricity, and a lack of perivesical and periprostatic fat may make the patient more susceptible to electric trauma. To self-induce a state of obesity in order to minimise the said risk is not recommendable due to the many and well-known disadvantages of obesity. The bladder wall is mainly muscle and as such a reasonably good conductor, muscle conducts electricity better in direction longitudinal to its fibres, in the order of some 0.67 to 0.85 S/m. The muscle conductivity in the transverse direction is 0.13 S/m. (S. Salinari, A. Bertuzzi, G. Mingrone, E. Capristo, A. Pietrobelli, P. Campion, A. V. Greco, and S. B. Heymsfield: Am J Physiol Endocrinol Metab Vol. 282, Issue 4, E960-E966, April 2002).

In the context of the invention, it should be noted that the bladder wall consists of several crisscrossing layers of muscle fibre but none of those layers or fibres transverse the bladder wall, so the in fact conductivity of the bladder wall in transverse direction is much closer to 0.13 S/m than that of the blood i.e. 0.67 S/m. Since a significant reduction of 50% in the proportion of electricity flowing through the muscle was achieved with making the irrigation fluid a better conductor than normal isotonic saline at 20° C. even when the direction of the current flow was longitudinal in respect to the muscle fibres, an even greater benefit is to be expected in vivo, where longitudinal conduction is not the rule. An irrigation solution with similar or compatible physiological properties and same or higher conductivity as blood would be ideal.

Table 1 gives examples on the conductivity of various tissues.

TABLE 1
Tissue conductivities

| Tissue | Tissue conductivity, S/m |
|---|---|
| Blood | 0.67 |
| Lung | 0.05 |
| Liver | 0.14 |
| Fat | 0.04 |
| Human trunk | 0.21 |
| Prostate | 0.46 |

By means of the invention, there is an improved conductivity between the electrodes inserted into the bladder or similar cavity inside the human or animal body. During transurethral surgery, based on electric current which achieves a plasmakinetic effect, the electrodes are constantly irrigated with an aqueous medium, and in the present invention, the conventional saline composition is replaced with an electrically conductive fluid having an electrical conductivity of at least 25 mS/cm at room temperature and typically being hypertonic.

It is preferred that the electrical conductivity of the solution is at least 28 mS/cm at room temperature.

The increase conductivity/tonicity can be achieved by increasing the level of alkaline metal ions and earth alkaline metal ions and mixtures thereof in the fluid. It is preferred to have a relatively high concentration of sodium ions optionally in mixture with potassium, calcium and magnesium ions or mixtures thereof. The sodium ions and potentially other cations, such as magnesium, are added in the form of salt having anions selected from the group of halogenide, carbonate and sulphate ions and anions of organic acids and mixtures thereof.

According to a preferred embodiment of the invention, the electrically conductive aqueous fluid for use in a method of surgery on the human or animal body, comprises a combination of sodium and magnesium ions.

The anions of the fluid (e.g. irrigation fluid) are in particular selected from the group of chloride ions, sulphate ions and mixtures thereof.

The fluid is formulated for use as an irrigation fluid in transurethral and transcutaneous surgery and diagnostic procedures. Such irrigation fluids may contain other components, such as amino acids and sugars. Examples include L-arginine, glycine and mannitol and sorbitol. Usually the amino acids and the sugars are used in concentrations of about 0.1 to 3.0%, typically about 1.0 to 2.0% by weight of the total composition.

The compositions are typically aqueous solutions which are used as such. However, naturally, depending on the actual use, it is possible to complement the composition with components commonly used in pharmacology, such as conventional auxiliary agents, including components for adjusting pH (e.g. mineral or organic acids, such as phosphoric acid, lactic acid, tartaric acid and gluconic acid and mixtures and salts thereof), tonicity modifiers, complexation-enhanging agents, and stabilizers. Generally, the concentration of the auxiliary agents of the above kind is up to about 5% by weight of the total composition.

It is also possible to add ethanol to the irrigation composition so that the total concentration thereof is about 0.1 to 3.0% by weight.

The pH of the irrigation liquid is typically about 4.5 to 7.0.

According to one embodiment, an irrigation solution is provided which has similar or compatible physiological properties as blood. According to another embodiment, the irrigation solution has the same conductivity as blood or higher.

It is known that bladder cancer cells, unlike normal urothelium, allow for the passage of salts and medication thought the cell membrane. Subjecting the cancer cells to irrigations of varying osmolality during the treatment may well have an additive effect to the other treatment modalities like mithomycin.

One embodiment according to the invention concerns a multi chamber infusion bag comprising a plurality of compartments for containing liquids, wherein at least one of which is filled with a fluid as discussed above, or wherein two or more compartments being filled with fluid which can be combined to provide a fluid of the above type.

Typically, the concentration of cations in the solution is as follows:

Na: 160 to 200 mol/l
K: 0 to 20 mol/l
Mg: 0 to 10 mol/l, in particular about 0.5 to 10 mol/l
Ca: 0 to 5 mol/l

Anions are present in amount corresponding to the those of the cations, since the cations are preferably added in the form of soluble salts, e.g. chloride, sulphate or mixtures of chloride and sulphate salts. Typically the concentration of the anion is about 160 to 235 mol/l.

The following examples will elucidate the invention:

### Example 1

First, a set of different electrolyte solutions were prepared and their conductivity measured in an SFS-EN ISO/IEC 17025 accredited commercial laboratory with the following results (Table 2)

TABLE 2
Conductivity of different electrolyte solutions

| | | | | Conductivity |
|---|---|---|---|---|
| | | Electrolytes g/liter of water | | mS/cm, at room |
| | | NaCl | MgSO₄ | temperature |
| 1 | Ringer-acetate | 3.2 | 1.85 | 18.9 |
| 2 | Modified Ringer- | 3.2 | 3.5 | 19.5 |
| | acetate | | | |
| 3 | Hypertonic solution | 18 | 1.85 | 30.8 |
| 4 | Hypertonic solution | 18 | 3.5 | 32.5 |
| 5 | Plain Ringer-acetate | | | 18.3 |
| 6 | Saline | 9 | | 16.5 |
| 7 | Modified Saline | 9 | 1.85 | 17.8 |
| 8 | Hypertonic solution | 18 | | 29.9 |

Raising the content of sodium chloride in the solution proved to be the best way to increase the conductivity. Adding of magnesium sulphate did little to achieve the same goal. The blend of Ringer—acetate and sodium chloride and magnesium sulphate, Samples 1 and 2, which contained 9.1 g of NaCl/litre showed a very similar result to the mixture of plain 9 g of NaCl and MgSO₄/litre, but might have other beneficial properties when in clinical use.

With regard to the use of magnesium, the following can be pointed out: Magnesium has been used in irrigation solutions in orthopedic operations due to its tendency to reduce post-operative pain. In the past, before the routine of sytostatic post-operative irrigation of the bladder, hypotonic solutions have been used to rinse the bladder after TURB in order to weaken and/or to cause a rupture of cancer cells remaining in the bladder. The effect of a continuous hypertonic irrigation during the operation is unknown. It is known that bladder cancer cells, unlike normal urothelium, allow for the passage of salts and medication thought the cell membrane. Subjecting the cancer cells to irrigations of varying osmolality during the treatment may well have an additive effect to the other treatment modalities like mithomycin. But this is only one possibility, and the invention is not limited to the use of magnesium as such or to the achievement of any particular effects with the aid of magnesium.

### Example 2

Secondly, a safety and feasibility test was performed: a sterile solution with 18 g of sodium chloride and 3.5 g of magnesium sulphate per litre of water was prepared and a healthy male volunteered to fill his bladder with 500 ml of said solution using a disposable single use 14ch catheter (Lofric®) to first empty the bladder completely and to fill it with the prepared solution.

The 0 hour blood sample was drawn 30 min before the bladder filling took place and the 1 hour and the 2 hour samples after respective time from the filling. The bladder was emptied by normally voiding after the 1 hour sample was drawn. The samples were analysed in a SFS-EN ISO/IEC 17025 accreted clinical laboratory.

During the bladder filling or the storage phase of the solution no adverse effects, such as pain or nausea or burning sensation, were observed apart from the slight discomfort of inserting the catheter. Nor were there any measurable changes in blood electrolytes during the filling and storing or the following 1 hour follow-up. Table 2 shows the blood test results.

TABLE 2
Blood electrolytes at given hour

| | | |
|---|---|---|
| 0 h | 1 h | 2 h |

| | | | | | | |
|---|---|---|---|---|---|---|
| Na | 141 | mmol/l | 141 | mmol/l | 141 | mmol/l |
| K | 4.1 | mmol/l | 4.1 | mmol/l | 4.1 | mmol/l |
| Mg | 0.85 | mmol/l | 0.84 | mmol/l | 0.84 | mmol/l |
| Krea | 80 | mmol/l | 81 | mmol/l | 82 | mmol/l |

### Example 3

Thirdly, a measuring of the change of current flow in a sample of tissue versus different solutions connected with it was arranged. In this ex vivo test a strip of fresh (not frozen—thawed) bovine muscle was used to simulate the perivesical and periprostatic tissue. It is to be understood that these conditions do not respond exactly to conditions and tissue behaviour in a real in vivo situation and should not be used as foundation to any new treatment modality without appropriate further research as stated by local laws. The test was made to demonstrate the soundness of the idea that increase of the conductivity of the irrigation solution does lessen the amount of current flow though the patient's tissue. A strip of muscle 15 cm long and 1.5 cm of both width and thickness was connected in parallel manner with a 500 ccm pool of irrigation solution, which electrolyte content was changed as can be seen in Column 1 in Table 3. Columns 2 and 3 show the corresponding amounts of current passed through the irrigation fluid and the tissue sample correspondingly. Finally in Column 4 the effect of changing the conductivity of irrigation fluid can be seen.

TABLE 3
Current through fluid vs. tissue

| | | Current | Current | |
|---|---|---|---|---|
| | | through | through | Tissue/fluid |
| | Irrigation fluid | fluid, A | tissue, A | ratio, % |
| 1 | Aqua ster. | 0 | 0.046 | 100 |
| 2 | NaCl 0.45 | 0.147 | 0.035 | 23.8 |
| 3 | NaCl 0.9 | 0.26 | 0.031 | 11.9 |
| 4 | NaCl 0.9 + MgSO₄ 3.5 | 0.284 | 0.029 | 10.2 |
| 5 | NaCl 1.8 | 0.412 | 0.027 | 6.6 |
| 6 | NaCl 1.8 + MgSO₄ 1.85 | 0.43 | 0.025 | 5.8 |
| 7 | NaCl 1.8 + MgSO₄ 3.5 | 0.432 | 0.025 | 5.8 |
| 8 | Aqua ster. + MgSO₄ 3.5 | 0.043 | 0.039 | 90.7 |

The amounts of the salts are indicated in wt-% of the solution

1. In a method of performing surgery or diagnosis on the human or animal body in a normal body cavity or in a cavity purpose-made in the human or animal body, wherein a tissue is cut using a first active cutting electrode and a second neutral electrode, both electrodes being immersed in an electrically conductive aqueous fluid and connected to a power source for applying a voltage and a current between the electrodes, said improvement comprising using a fluid having an electrical conductivity of at least 25 mS/cm at room temperature.

2. The method according to claim 1, wherein the aqueous fluid has an electrical conductivity of at least 28 mS/cm at room temperature.

3. The method according to claim 1, wherein the aqueous fluid comprises cations selected from the group of alkaline metal ions and earth alkaline metal ions and mixtures thereof.

4. The method according to claim 3, wherein the aqueous fluid comprises sodium ions optionally in mixture with potassium, calcium and magnesium ions or mixtures thereof.

5. The method according to claim 1, wherein the aqueous fluid comprises anions selected from the group of halogenide, sulphate and carbonate ions and anions of organic acids and mixtures thereof.

6. The method according to claim 1, wherein the electric energy applied by the cutting electrode is at least 80 W, preferably about 100 to 350 W, in particular about 100 to 320 W.

7. The method according to claim 1, wherein the aqueous fluid is hypertonic.

8. The method according to claim 1, wherein the surgery comprises transurethral or transcutaneous surgery on the human or animal body.

9. A method of performing cancer therapy on the human or animal body in a body cavity, wherein cancer tissue is being irrigated with an electrically conductive aqueous fluid, said fluid having an electrical conductivity of at least 25 mS/cm at room temperature.

10. The method according to claim 9, wherein the aqueous fluid has an electrical conductivity of at least 28 mS/cm at room temperature.

11. The method according to claim 9, wherein the aqueous fluid comprises cations selected from the group of alkaline metal ions and earth alkaline metal ions and mixtures thereof.

12. The method according to claim 11, wherein the aqueous fluid comprises sodium ions optionally in mixture with potassium, calcium and magnesium ions or mixtures thereof.

13. The method according to claim 9, wherein the aqueous fluid comprises anions selected from the group of halogenide, sulphate and carbonate ions and anions of organic acids and mixtures thereof.

14. The method according to claim 9, wherein the aqueous fluid is hypertonic.

15. The method according to claim 9, wherein the treatment is carried out in conjunction with other treatment modalities, like mithomycin.

16. An electrically conductive aqueous fluid for use in a method of surgery or diagnosis on the human or animal body, wherein a tissue is cut using a first active cutting electrode and a second neutral electrode, both electrodes being immersed in the electrically conductive aqueous fluid and connected to a power source for applying a voltage and a current between the electrodes, comprising a fluid having an electrical conductivity of at least 25 mS/cm at room temperature.

17. The fluid according to claim 16, wherein the electrical conductivity is at least 28 mS/cm at room temperature.

18. The fluid according to claim 16, comprising cations selected from the group of alkaline metal ions and earth alkaline metal ions and mixtures thereof.

19. The fluid according to claim 18, comprising sodium ions optionally in mixture with potassium, calcium or magnesium ions or mixtures thereof.

20. The fluid according to claim 16, comprising anions selected from the group of halogenide, sulphate and carbonate ions and anions of organic acids and mixtures thereof.

21. The fluid according to claim 16, wherein the aqueous fluid is hypertonic.

22. The fluid according to claim 16, wherein the fluid is formulated for use as an irrigation fluid in transurethral and transcutaneous surgery and diagnostic procedures.

23. A multi chamber infusion bag comprising a plurality of compartments for containing liquids, wherein at least one of which is filled with a fluid according to claim 16 or wherein two or more compartments are filled with fluid which can be combined to provide a fluid according to claim 16.
